(19) United States
(12) Patent Application Publication
(10) Pub. No.: US 2008/0194801 A1
(43) Pub. Date: Aug. 14, 2008

(54) Robust multidentate ligands for diagnosis and anti-viral drugs for influenza and related viruses

(76) Inventors: Basil I. Swanson, Los Alamos, NM (US); Jurgen G. Schmidt, Los Alamos, NM (US); Suri S. Iyer, Cincinnati, OH (US); Ramesh R. Kale, Cincinnati, OH (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 11/728,334

(22) Filed: Mar. 26, 2007

Related U.S. Application Data
(60) Provisional application No. 60/901,516, filed on Feb. 14, 2007.

(51) Int. Cl. C07H 15/00

(52) U.S. Cl. 536/17.4

(57) ABSTRACT

Design and synthesis of a novel library of compounds comprising a spacer with an attachment element on one terminus and a recognition element on the other terminus is presented. The library of compounds can be attached to a solid support and used as an integral component of sensors and biosensors or the library of compounds can be used as antiviral drugs or to isolate pathogens from complex mixtures for further analysis.

### RELATED APPLICATIONS

This application claims the benefit of U.S. Provisional Application 60/901,516, filed on Feb. 14, 2007.

### STATEMENT OF FEDERAL RIGHTS

The United States government has rights in this invention pursuant to Contract No. DE-AC51-06NA25396 between the United States Department of Energy and Los Alamos National Security, LLC for the operation of Los Alamos National Laboratory.

### FIELD OF INVENTION

The present invention relates to a compound that can be used as an anti-viral drug to counter infections from influenza and other viruses. More specifically, the compound is based on multidentate ligands that target the natural receptor sites on the surface of viral particles.

### BACKGROUND

N-acetyl neuraminic acid (i.e., sialic acid) is a structurally unique nine-carbon keto sugar that is the terminal carbohydrate residue of several surface glycoproteins and glycolipids of mammalian cells. Several microbes, including the opportunistic influenza virus, use sialic acid for cellular entry and infection. Because the binding sites are highly conserved for influenza virus, sialic acid derivatives are extremely important for the development of antivirals.

All influenza variants, including the highly pathogenic H5N1 species, have two cell surface proteins, hemagglutinin (“HA”) and neuraminidase (“NA”), that mediate recognition and binding to the host cell. The optimal binding between a particular strain and the host cell is highly dependent on specific structural features and the density of the sialic acid derivatives. For example, the influenza C virus specifically infects (i.e., binds to) cells that display p-O-acetyl sialic acids, whereas influenza A and B do not.

In addition to the terminal sialic acid, the nature and structure of the penultimate sugars appear to play a critical role in defining the binding efficiency of the microbe to the host cell. Several studies show that avian and human influenza prefer sialic acids linked to the three and six positions of galactose, respectively. M. N. Matrosovich et al., Proc. Nat. Acad. Sci., 2004, 101, 4620-24; M. N. Matrosovich et al., Influenza Virol., 2006, 95-137. This preferential recognition has significant implications from a viral transmission viewpoint. For example, the upper respiratory track of humans is rich in α-2,6 sialic acid linked glycans, whereas cells in the lower respiratory tract display increasing numbers of terminal α-2,3 linkages. In contrast, the respiratory and intestinal tracts of fowl predominantly comprise α-2,3 sialic acids. This difference may explain the dominance of bird-to-human as opposed to human-to-human H5N1 viral transmissions.

Glycan microarrays also contribute to the understanding of receptor specificities of HA variants. Subtle structural nuances of sialoligosaccharides, such as O-sulfation at the specific locations, influence the binding affinity tremendously. Even though these studies are critical, it is important to note that most of these studies use natural oligosaccharides and some synthetic glycans. Because batch-to-batch variations, undesirable contaminants, and infectious agents frequently plague carbohydrates from biological sources, synthetic analogues are important. In the case of influenza, naturally occurring sialic acid derivatives as stable ligands for hand held biosensor applications are not ideal because the viral NA cleaves the innate O-glycoside for subsequent infection. In addition to stability and positional isomerism, other factors such as orientation of the sugars, mono/multivalency, tether length, choice of scaffold, and ancillary groups dictate the binding efficiency.

A modular synthetic approach that addresses most, if not all, of these variables to yield a library of compounds could be very useful in the development of drugs or as biological reagents. In addition, glycoconjugates on solid supports could be used as integral components of sensors or biosensors or to isolate pathogens from complex mixtures for further analysis.

### SUMMARY OF THE INVENTION

The present invention discloses novel compounds comprising a flexible spacer with an attachment element on one terminus and a recognition element on the other terminus. The compound comprises (a) a flexible spacer having a first terminus and a second terminus, (b) an attachment element connected to said first terminus and comprising a di-, tri-, tetra-, or multivalent scaffold and that is capable of either (i) providing an output signal, or (ii) attaching to a substrate, membrane, or a magnetic bead; and (c) a recognition element connected to said second terminus that is capable of attaching to (i) a HA, (ii) a NA, or (iii) a HA or a NA attached to an intact organism.

A possible embodiment of the flexible spacer includes oligoethylene glycol (“OEG”). The length of the OEG can vary from 3 to 21 repeating units. The recognition elements can be glycoconjugates, peptides, or a combination of molecules. Moreover, the recognition element can contain functional groups independently selected from the group consisting of an amine, a guanidium group, a sulfate, a carbohydrate, and a peptide. A possible embodiment of the attachment element includes a biotinylated scaffold. Moreover, the attachment element can attach to a membrane, a self-assembled monolayer, a waveguide, a magnetic bead, a protein, a solid phase, or an anchor.

Possible embodiments of the novel compounds are shown in FIGS. 4, 6, and 8.

### BRIEF DESCRIPTION OF THE DRAWINGS

FIG. 1 shows a model of the three element divalent compound.

FIG. 2 shows the synthesis of the biotinylated scaffold.

FIG. 3 shows the synthesis of the α-2,6 analogue.

FIG. 4 shows a possible embodiment of the α-2,6 analogue.

FIG. 5 shows the synthesis of the dimeric S-sialoside.

FIG. 6 shows a possible embodiment of the dimeric S-sialoside.

FIG. 7 shows the synthesis of the tetravalent S-sialoside.

FIG. 8 shows a possible embodiment of the tetravalent S-sialoside.

### DETAILED DESCRIPTION

The claimed invention is a compound that can be used as an anti-viral drug to counter infections from influenza and other viruses. The claimed compounds bind to viral surface proteins to either block cellular invasion or inhibit enzymatic activity. The overall binding between a particular virus strain and a host cell is highly dependent on structural features of the sialic acid derivatives and the density of the sugar residues. In addition, the binding efficiency of influenza virus variants depends on the penultimate sugars. For example, avian and human influenza viruses respectively target 2,3 and 2,6 linked sialic acids and structural variants thereof.

Recognizing these binding dependencies, the claimed compounds comprise a spacer with an attachment element on one terminus and a recognition element on the other terminus. The claimed compounds serves as a “pattern of recognition” system where synthetic surface sugars of a cell presented in a suitable format generate unique fingerprint patterns upon exposure to various viruses. The claimed approach also conserves the host cell's surface glycoproteins for binding so that emerging pathogenic and drifting virus strains can bind to the library of compounds.

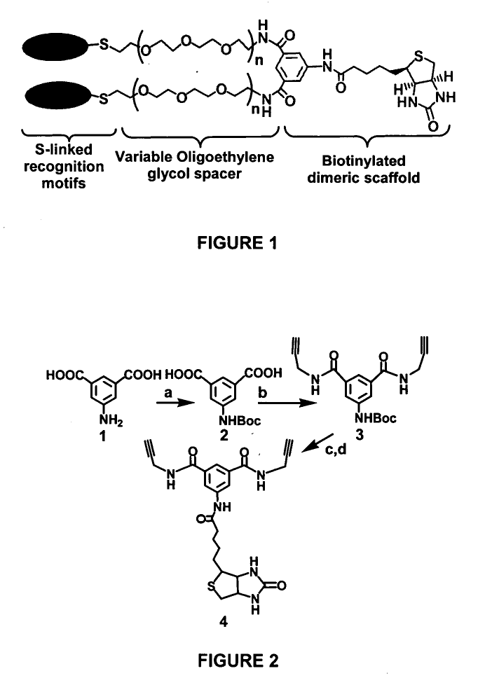

A possible embodiment of the compound is shown in FIG. 1 and contains three important components: (i) an attachment element such as a biotinylated divalent scaffold; (ii) a recognition element such as an S-sialoside; and (iii) a flexible spacer that connects and separates the attachment element from the recognition element such as OEG. Each component is individually discussed below.

The attachment element may be any functional group capable of either (i) providing an output signal or (ii) attaching to a substrate, membrane, or a magnetic bead. In one embodiment the attachment element is a biotinylated divalent scaffold. The scaffold may be di-, tri-, tetra-, or multivalent to increase avidity. The rationale for using biotin is that the avidin-biotin system is well studied and characterized. Moreover, biotin affords multivalency as avidin binds four biotin molecules. Further, avidin coated magnetic beads and fluorescent nanoparticles are commercially available for biotin coupling.

The recognition element may be any material capable of attaching to (i) a HA, (ii) a NA, or (iii) an intact organism attached to a HA or NA. The following prior art, herein incorporated by reference, teaches acceptable recognition elements: Babu et al (U.S. Pat. No. 5,602,277; U.S. Pat. No. 6,410,594; U.S. Pat. No. 6,562,861); Bischofberger et al (U.S. Pat. No. 5,763,483; U.S. Pat. No. 5,952,375; U.S. Pat. No. 5,958,973); Brouillette et al (U.S. Pat. No. 6,509,359); Kent et al (U.S. Pat. No. 5,886,213); Kim et al (U.S. Pat. No. 5,512,596); Lew et al (U.S. Pat. No. 5,866,601); Luo et al (U.S. Pat. No. 5,453,533); and von Izstein (U.S. Pat. No. 5,360,817). Because the recognition element is only capable of capturing one strain, specificity is possible with the synthetic recognition elements. The recognition element could be a glycoconjugate, a peptide, or a combination of molecules. The recognition element may be a N-sialoside or a C-sialoside. In one embodiment the recognition element is an S-sialoside. The rationale for using an S-sialoside is that an S-sialoside shows improved stability without impacting binding affinities. Moreover, NA does not easily cleave the S-glycoside bond.

The flexible spacer may be any material capable of connecting and separating the attachment element from the recognition element. The flexible spacer can be tailored to the necessary length so that the recognition element can reach a binding site. The length can be as short as a single atom or it may be longer. Examples of the flexible spacer include, but are not limited to, amides, linear polyethers, and ringed polyethers. In one embodiment the flexible spacer is OEG. The rationale for using OEG is that OEG reduces non-specific binding and imparts a degree of flexibility so the recognition element can attain the proper orientation for a tighter fit. Moreover, OEG allows variable spacer lengths to optimize sensor response and minimize unspecific interaction of the analyte with the membrane surface.

The library of compounds can be attached to a solid support and used as integral components of sensors or biosensors or the library of compounds can be used to isolate pathogens from complex mixtures for further analysis. The compound could be attached to a membrane, a self-assembled monolayer, a waveguide, a magnetic bead, a protein, a solid phase, or an anchor by processes known to those skilled in the art. For example, if the recognition element is biotin, then the biotinylated compound can be gently shaken with streptavidin coated magnetic beads until the bead is completely saturated with the ligands. After thirty minutes, the excess ligand can be washed away using a phosphate buffer saline (“PBS”). See, e.g., Ismail H. Boyaci et al., Amperometric determination of live Escherichia coli using antibody-coated paramagnetic beads, Anal Bioanal Chem (2005) 382: 1234-41.

Reference is now made in detail to four examples. The first example teaches the synthesis of the biotinylated scaffold shown in FIG. 2. The biotinylated scaffold can be prepared by protecting the amine group of 1 with a tert-butoxycarbonyl (“t-Boc”) derivative to yield 2. Reacting the acid functionalities with propargyl amine yielded 3. Removal of the protecting group, followed by 2,4-dichloro-6-methoxy-1,3,5-triazine (“CDMT”)/N-methylmorpholine (“NMM”) mediated coupling, yielded 4. The alkyne and the biotin rings were confirmed by high resolution mass spectrometry (“HRMS”). The synthesized scaffold can be used for 1,3 dipolar bioconjugation to azide containing biomolecules.

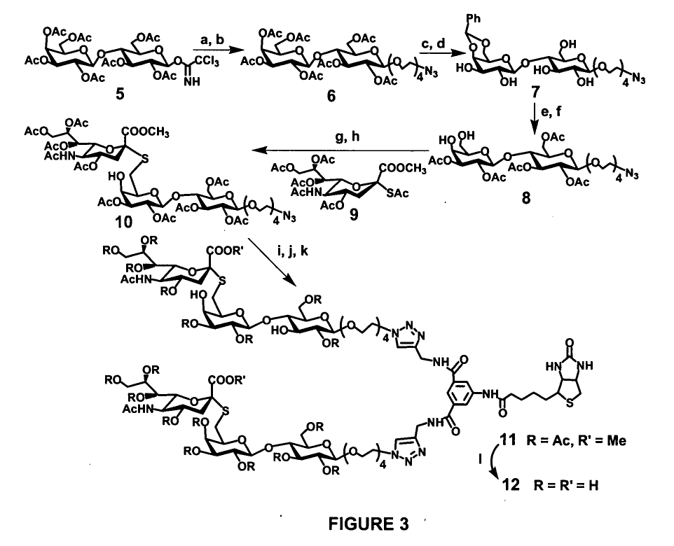

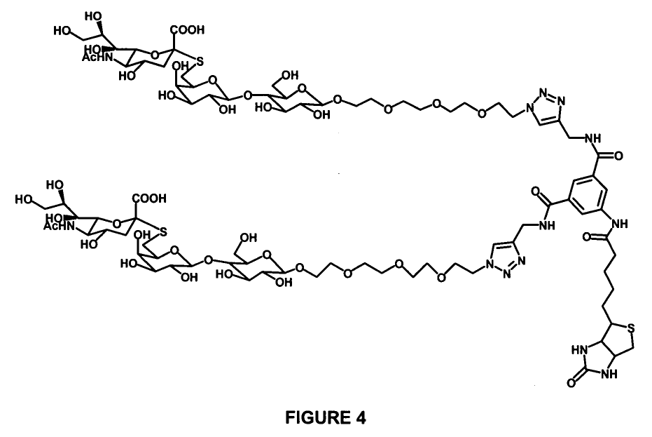

The second example teaches the synthesis of the α-2,6 analogue shown in FIG. 3. The α-2,6 analogue may have the configuration shown in FIG. 4. The α-2,6 analogue can be prepared by reacting 1-azido-(2-(2-ethoxy)ethoxy)ethanol with 5 to yield the beta isomer 6. NMR indicated a beta-coupled product. Saponification using sodium methoxide (“NaOMe”) in methanol (“MeOH”) was followed by benzylidene protection of the 4,6 hydroxyl groups to yield 7. Reprotection of the free hydroxyl groups with acetic anhydride in the presence of pyridine followed by removal of the ketal yielded 8. The primary alcohol in 8 was selectively activated to its triflate (“OTf”) and reacted with the known thio-N-acetylneuraminic acid 9 in the presence of diethyl amine to yield 10. HRMS confirmed the product's existence. Next, 10 and 4 were reacted in the presence of copper (II) sulfate (“CuSO₄”) and ascorbic acid in a water/tetrahydrofuran (“THF”) mixture to yield 11. A two-step procedure was required to remove the protecting groups, so saponification followed by deesterification yielded 12. The final product was purified using a Biogel P-2 gel column with water as eluent.

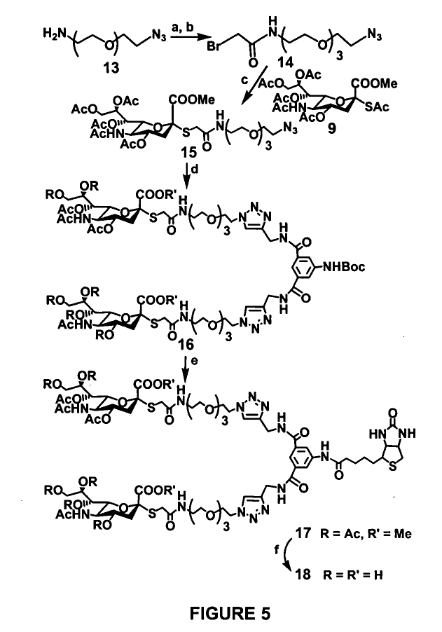

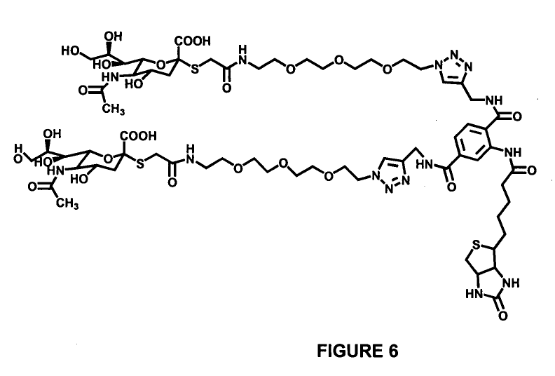

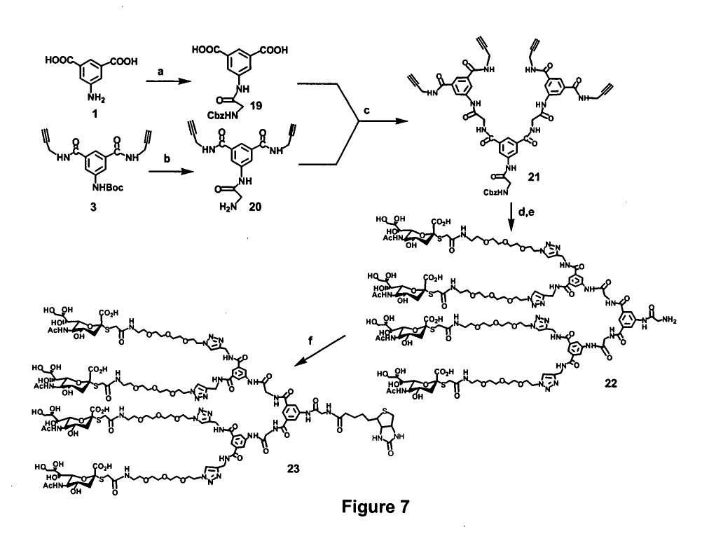

The third example teaches the synthesis of the dimeric S-sialoside shown in FIG. 5. The dimeric S-sialoside may have the configuration shown in FIG. 6. The dimeric S-sialoside can be prepared by derivatizing tetraethylene glycol monoamine monoazide 13 with bromoacetyl bromide to yield 14. Thio-N-acetylneuraminic acid 9 was reacted with the bromide in the presence of diethyl amine to yield 15. HRMS confirmed the existence of the thioether bond. Reacting 15 with the dimeric scaffold 3 yielded 16. Removal of the t-Boc protecting group, followed by activation using CDMT, NMM, and 5-carboxyl biotin, yielded the biotinylated product 17. Next, 17 was subjected to saponification and deesterification. The final dimeric S-sialoside 18 was purified using a Biogel B-10 column to yield a white foam.

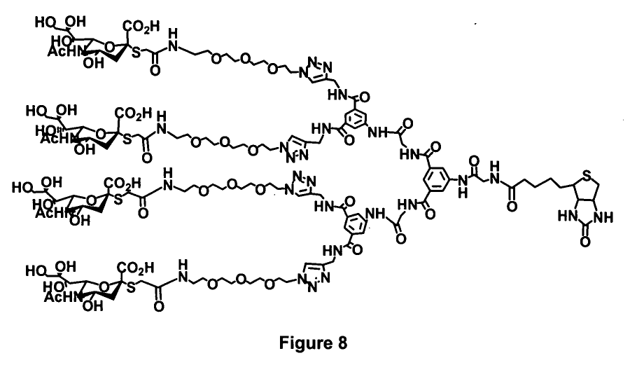

The fourth example teaches the synthesis of the tetravalent S-sialoside shown in FIG. 7. The tetravalent S-sialoside may have the configuration shown in FIG. 8. The tetravalent S-sialoside can be prepared by reacting 1 with chloroacetyl chloride. The mixture was subsequently reacted with aqueous ammonia to yield an amine. The amine formed was protected with a CBz group to yield 19. Deprotection of the t-Boc group in 3, followed by sequential treatment with bromoacetyl bromide and methanolic ammonia, yielded amine 20. Two equivalents of 20 were reacted with the carboxylic acid residues of the amino protected isophthalic acid derivative 19 in the presence of CDMT/NMM to yield 21. Next, 21 and 9 were reacted in the presence of CuSO₄ and ascorbic acid in a water/THF mixture to yield a tetravalent-coupled product with acetate groups. A two-step procedure was required to remove the protecting groups, so saponification followed by deesterification yielded 22. Compound 22 was purified using a Biogel P-2 gel column with water as eluent. Reaction of 22 with 5, in the presence of CDMT and NMM, yielded 23.

All chemical reagents were of analytical grade and were used as supplied without further purification unless indicated. Acetic anhydride and acetyl chloride were distilled under an inert atmosphere and stored under argon. Four-angstrom molecular sieves were stored in an oven (greater than 130° C.) and cooled in vacuo. The acidic ion-exchange resin used was Dowex-50 and Amberlite (H⁺ form). Analytical thin layer chromatography (“TLC”) was conducted on silica gel 60-F254 (Merck). Plates were visualized under ultraviolet light and/or by treatment with acidic cerium ammonium molybdate followed by heating. Column chromatography was conducted using silica gel (230-400 mesh) from Qualigens. ¹H and ¹³C NMR spectra were recorded on Bruker AMX 400 MHz spectrometer. Chemical shifts were reported in δ (ppm) units using ¹³C and residual ¹H signals from deuterated solvents as references. Spectra were analyzed with Mest-Re-C Lite (Mestrelab Research) and/or XwinPlot (Bruker Biospin). Electrospray ionization mass spectra were recorded on a Micromass qTOF II (Waters) and data were analyzed with MassLynx 4.0 (Waters) software.

### EXAMPLE 1

### Synthesis of Biotinylated Divalent Scaffold

Step a. The biotinylated divalent scaffold can be prepared according to the scheme shown in FIG. 2 by mixing 1 (0.27 grams (“g”); 0.76 millimol (“mmol”)) with dry CH₂Cl₂ (10 milliliters (“mL”)) and excess di-t-butyl dicarbonate and triethylamine. The mixture was continuously stirred at room temperature for 2 hours (“h”). The reaction was quenched with water and the organic layer was extracted with methylene chloride (“CH₂Cl₂”) to yield 2 (0.35 g, 85%) as a solid.

Step b. CDMT (6.89 g; 39.15 mmol) and 2 (5 g; 17.8 mmol) were mixed in dry THF (20 mL) under stirring at 0° C. NMM (4.30 mL; 39.15 mmol) in THF (10 mL) was added drop wise to the mixture. The mixture was continuously stirred at 0° C. overnight. Propargyl amine (2.72 mL; 39.15 mmol) and NMM (4.30 mL; 39.15 mmol) in a DMF/THF solution (10 mL; 1:5) were added drop wise to the mixture under stirring at 0° C. The mixture was continuously stirred for 20 h with the reaction slowly warming to room temperature. The reaction was stopped by adding water drop wise to the mixture under stirring. The product was extracted with ethyl acetate (“EtOAc”) (25 mL). The organic layers were dried over anhydrous sodium sulfate (“Na₂SO₄”) and filtered. The solvent was removed in vacuo. The crude product was purified by flash column chromatography, eluting with a hexane/EtOAc solution (1:3), to yield 3 (5.37 g; 85%) as a white solid.

Step c. The white solid 3 was dissolved in dry CH₂Cl₂ (50 mL). Triisopropylsilane (0.16 mL; 0.76 mmol) was added to the mixture. Trifluoroacetic acid (0.56 mL; 7.60 mmol) was added drop wise to the mixture. The mixture was continuously stirred at room temperature for 8 h. The mixture was cooled to 0° C. The reaction was quenched with saturated sodium bicarbonate (“NaHCO₃”) and the product was extracted with CH₂Cl₂ (2×25 mL). The organic layer was dried over anhydrous Na₂SO₄ and filtered. The solvent was removed in vacuo. The crude product was purified by flash column chromatography, eluting with a hexane/EtOAc solution (1:4), to yield a pale yellow free amine solid (155 g; 80%).

Step d. Biotin (0.12 g; 0.49 mmol) and CDMT (0.10 g; 0.56 mmol) were mixed in a dry THF/DMF solution (6 mL; 1:1) under argon at 0° C. NMM (0.11 mL) in THF (1.0 mL) was added drop wise to the mixture. The mixture was continuously stirred at 0° C. for 12 h. In a separate flask the free amine (80 milligram (“mg”); 0.3 mmol) was dissolved in a DMF/THF solution (1 mL; 1:1). NMM (0.11 mL) was added to the flask. The flask mixture was added to the activated biotin at 0° C. and the mixture was reacted for 24 h with the reaction slowly warming to room temperature. The reaction was quenched with deionized water and the product was extracted with EtOAc (25 mL). The organic layer was dried over anhydrous Na₂SO4 to yield a soft off-white compound. The crude product was purified by flash column chromatography, eluting with an EtOAc/MeOH solution (85:15), to yield 4 (0.12 mg; 78%) as a white solid.

### EXAMPLE 2

### Synthesis of the α-2,6 Analogue

Step a, b. The α-2,6 analogue can be prepared according to the scheme shown in FIG. 3 by dissolving 5 (7.68 g; 9.83 mmol) and 1-azido-(2-(2-ethoxy)ethoxy)ethanol (2.68 g; 12.2 mmol) in CH₂Cl₂ (50 mL) and cooling to 0° C. A solution of trimethylsilyl trifluoromethanesulfonate (“TMSOTf”) in CH₂Cl₂ (8.9 mL; 0.22 mol; 0.2 equiv.) was added drop wise to the mixture. The mixture was continuously stirred at 0° C. for 1.5 h. The reaction was quenched with a cold saturated solution of NaHCO₃ and the product was extracted with CH₂Cl₂. The organic layer was dried over anhydrous Na₂SO₄ and filtered. The filtrate was concentrated in vacuo. The crude product was purified by flash column chromatography, eluting with a hexane/EtOAc solution (3:7), to yield 6 (5.77 g; 70%) as a sticky white solid.

Step c. MeOH (30 mL) and 6 (4.820 g; 5.75 mmol) were mixed. NaOMe in MeOH (0.7 molar (“M”); 2 mL) was added drop wise to the mixture. The mixture was continuously stirred at room temperature for 5 h. The reaction was quenched with a careful addition of Dowex H⁺ resin (pH≈6) and the resin was filtered. The solvent was removed in vacuo. The crude product was purified by flash column chromatography, eluting with a CH₂Cl₂/MeOH solution (4:1), to yield a white solid (3.205 g; 85%).

Step d. The white solid (2.0 g; 3.67 mmol) was dissolved in anhydrous acetonitrile (“CH₃CN”) (20 mL). Benzaldehyde dimethyl acetal (1.39 mL; 5.51 mmol) was added to the mixture under argon. p-Toluenesulfonamide (“p-TSA”) (100 mg; 0.53 mmol) was added to the mixture. The mixture was continuously stirred at room temperature for 16 h. The reaction was quenched with triethyl amine. The solvent was removed in vacuo. The crude product was purified by flash column chromatography, eluting with a CH₂Cl₂/MeOH solution (9:1), to yield 7 (2.04 g; 88%) as a white solid.

Step e. Dry pyridine (15 mL) and 7 (1.5 g; 2.37 mmol) were mixed. A catalytic amount of 4-dimethylaminopyridine (“DMAP”) (50 mg; 0.41 mmol) was added to the mixture. Acetic anhydride (3 mL) was added drop wise to the mixture at 0° C. The mixture was continuously stirred at 0° C. for 16 h. The solvent was removed in vacuum. The residue was dissolved in CH₂Cl₂ and sequentially washed with hydrochloric acid (“HCl”) (1 M), saturated NaHCO₃, and water. The organic layer was dried over anhydrous Na₂SO₄. The solvent was removed in vacuo. The crude product was purified by flash column chromatography, eluting with an EtOAc/hexane solution (9:1), to yield a white solid (1.80 g; 90%).

Step f. The white solid (1.80 g) was dissolved in CH₂Cl₂ (50 mL). The mixture was cooled to 0° C. A trifluoracetic acid/water solution (3:2; 55 mL) was added drop wise to the mixture under argon. The ice bath was removed for 1 h. The mixture was diluted with CH₂Cl₂ (50 mL). The reaction was quenched with cold saturated NaHCO₃ (100 mL). The organic layer was dried over anhydrous Na₂SO₄. The solvent was removed in vacuo. The crude product was purified by flash column chromatography, eluting with an EtOAc/hexane solution (9:1), to yield 8 (1.46 g; 75%) as a white solid.

Step g. Dry CH₂Cl₂ (10 mL) and 8 (200 mg; 0.26 mmol) were mixed. Pyridine (86 microliters (“μL”); 1.061 mmol) was added to the mixture under argon. The entire mixture was cooled to −25° C. Trifluoromethanesulfonic anhydride (53 μL; 0.32 mmol) was added drop wise to the mixture. The mixture was continuously stirred at room temperature for 1 h. After TLC indicated complete disappearance of the starting material, the mixture was sequentially washed with HCl (1 N), saturated NaHCO₃, and water. The organic layer was dried over anhydrous Na₂SO₄ and filtered. The solvent was removed in vacuo. The activated primary alcohol was used without further purification.

Step h. Thio-N-acetylneuraminic acid 9 (175 mg; 0.318 mmol) was added to the triflate. Dry DMF (8 mL) was added to the mixture. The mixture was cooled to −25° C. Diethyl amine (0.27 mL; 2.65 mmol) was added drop wise to the mixture. The mixture was continuously stirred at room temperature for 2 h. The solvent was removed in vacuo. The crude product was purified by flash column chromatography, eluting with an EtOAc/MeOH solution (95:5), to yield 10 (215 mg; 65%) as a white solid.

Step i, j, k. CuSO₄ (2 mg; 0.008 mmol) in a water/THF solution (3 mL; 1:1), 4 (4 mg; 0.0083 mmol), 10 (23 mg; 0.018 mmol), and sodium ascorbate (4 mg; 0.206 mmol) were mixed. The mixture was continuously stirred at room temperature for 36 h. The solvent was removed under vacuum. The crude product was purified by flash column chromatography, eluting with a CH₂Cl₂/MeOH solution (75:25), to yield 11 (16 mg; 67%) as a white solid. HRMS calculated for [C₁₂₄H₁₇₅N₃O₆₄S₃+2H⁺]²⁺=1484.0073. Found 1484.1948.

Step l. MeOH (1.0 mL) and 11 (12 mg; 4 micromol (“μmol”)) were mixed. NaOMe in MeOH (0.5 mL; 0.7 M) was added drop wise to the mixture. The mixture was continuously stirred at room temperature for 12 h. The solvent was removed in vacuo. The residue was dissolved in aqueous sodium hydroxide (“NaOH”) (2 mL; 0.05 N) and continuously stirred at room temperature for 10 h. The reaction was quenched with a careful addition of Amberlite H+ resin (pH≈6). The resin was filtered and the solvent was removed in vacuum. The crude product was purified by size exclusion chromatography using Biogel P-2 gel. The crude product was lyophilized to yield 12 (8 mg; 91%) as a white solid. HRMS calculated for [C₈₆H₁₃₅N₁₃O₄₆S₃+2H⁺]²⁺=1091.8966. Found 1091.9143.

### EXAMPLE 3

### Synthesis of the Dimeric S-sialoside

Step a. The dimeric S-sialoside can be prepared according to the scheme shown in FIG. 5 by dissolving 13 (1.07 g; 4.20 mmol) in anhydrous CH₃CN (15 mL). See, e.g., A. W. Schwabacher et al, Desymmetrization reactions: efficient preparation of unsymmetrically substituted linker molecules, J. Org. Chem., (1998), 63, 1727-29. Sodium carbonate (“Na₂CO₃”) (2.23 g; 21 mmol) was added to the mixture. The mixture was continuously stirred at room temperature for 12 h. EtOAc (20 mL) was added to the mixture. The mixture was filtered to remove the Na₂CO₃. The solvent was removed in vacuo to yield a free amine (0.92 g; 4.2 mmol).

Step b. The free amine (0.92 g; 4.2 mmol) was dissolved in anhydrous CH₃CN (15 mL). Na₂CO₃ (2.23 g; 21 mmol) was added to the mixture. The mixture was cooled to 0° C. Bromoacetyl bromide (0.44 mL; 5.0 mmol) in CH₃CN (6 mL) was added drop wise to the mixture. The mixture was continuously stirred at room temperature for 12 h. The mixture was diluted with EtOAc (25 mL) and filtered through celite to yield 14 (1.22 g; 80%) as a viscous oil. HRMS calculated for [C₁₀H₂₀BrN₄O₄]=339.0668. Found 339.0592.

Step c. Thio-N-acetylneuraminic acid 9 (170 mg; 0.31 mmol) was dissolved in DMF (4 mL). A solution of 14 (105 mg; 0.31 mmol) in DMF (4 mL) was added to the mixture. Diethylamine (“Et₂NH”) (2 mL) was added drop wise to the mixture. The mixture was continuously stirred at room temperature for 10 h. Excess Et₂NH and DMF were removed in vacuo. The crude product was purified by flash column chromatography, eluting with a CH₂Cl₂/MeOH solution (95:5), to yield 15 (218 mg; 92%) as a viscous oil. HRMS calculated for [C₃₀H₄₈N₅O₁₆S]=766.2817. Found 766.2814.

Step d. CuSO₄ (0.021 g; 0.086 mmol) in a water/THF solution (4 mL; 1:1), 4 (0.025 g; 0.071 mmol), 15 (0.12 g; 0.157 mmol), and sodium ascorbate (0.034 g; 0.171 mmol) were mixed. The mixture was continuously stirred at room temperature until the starting materials completely disappeared (approximately 24 h). The solvent was evaporated. The crude product was purified by flash column chromatography, eluting with an EtOAc/MeOH solution (8:2), to yield 16 (0.103 g; 78%) as a white, fluffy solid. HRMS calculated for [C₃₀H₄₈N₅O₁₅S (M+H)]=766.2817. Found 766.2814.

Step e. Compound 16 (60 mg; 0.032 mmol) was dissolved in dry CH₂Cl₂ (10 mL). Triisopropylsilane (0.02 mL) was added to the mixture. Trifluoroacetic acid (0.15 mL) was added drop wise to the mixture. The mixture was continuously stirred at room temperature for 12 h. The reaction was quenched with a saturated solution of NaHCO₃ (10 mL) and the amine was extracted with CH₂Cl₂ (5 mL). The organic layer was dried over anhydrous Na₂SO₄ and filtered. The solvent was removed in vacuo. The crude product was purified by flash column chromatography, eluting with an EtOAc/MeOH solution (3:1), to yield a white solid free amine (48 mg; 84%). The free amine was used directly without further purification.

Biotin (0.12 g; 0.49 mmol) and CDMT (0.10 g; 0.56 mmol) were mixed in a dry THF/DMF solution (6 mL; 1:1) under argon at 0° C. NMM (0.11 mL) in THF (1.0 mL) was added drop wise to the mixture. The mixture was stirred at 0° C. for 12 h. In a separate flask the free amine (48 mg; 0.027 mmol) was dissolved in a THF/DMF solution (1 mL; 1:1). NMM (0.11 mL) was added to the flask. The flask solution was added to the activated biotin at 0° C. The mixture was continuously stirred for 20 h with the reaction slowly warming to room temperature. The reaction was quenched with an aqueous HCl solution (0.1 N) added drop wise and the compound was extracted with EtOAc (25 mL). The organic layer was dried over anhydrous Na₂SO₄ and filtered. The solvent was removed in vacuo. The crude product was purified by flash column chromatography, eluting with a CH₂Cl₂/MeOH solution (8:2), to yield 17 (26 mg; 49%) as a white solid.

Step f. MeOH (1.0 mL) and 17 (20 mg; 0.010 mmol) were mixed. NaOMe in MeOH (0.5 mL; 0.7 M) was added drop wise to the mixture. The mixture was continuously stirred at room temperature for 12 h. The solvent was removed in vacuo. The residue was dissolved in aqueous NaOH (2 mL; 0.05 N) and continuously stirred at room temperature for 10 h. The reaction was quenched with a careful addition of Amberlite H+ resin (pH≈6). The resin was filtered. The solvent was removed in vacuo. The crude product was purified by size exclusion chromatography using Biogel P-2 gel. The crude product was lyophilized to yield 18 (15 mg; 92%) as a white solid. HRMS calculated for [C₈₄H₁₂₁N₁₅O₃₆S³+2H⁺]²⁺=1006.8703. Found 1006.8750.

### EXAMPLE 4

### Synthesis of the Tetravalent S-sialoside

Step a. The tetravalent S-sialoside can be prepared according to the scheme shown in FIG. 7 by dissolving 1 (11.3 g; 62.38 mmol) in NaOH (50 mL; 4 M). The mixture was cooled 0° C. Chloroacetyl chloride (17 mL; 214 mmol) was added drop wise to the mixture under continuous stirring. The mixture was continuously stirred at 0° C. for 20 minutes. Aqueous HCl (pH≈1.5) was added to the mixture and a white bulky precipitate formed. The precipitate was filtered, washed with cold water, and dried in vacuum to yield a bulky white solid (13.2 g; 82%). The solid was dissolved in a concentrated aqueous ammonia (“NH₃”) solution (200 mL) and continuously stirred at room temperature for 12 h. The mixture volume was reduced to approximately 30 mL. Ethanol (“EtOH”) (30 mL) was added to the mixture. The mixture was cooled and a white precipitate formed. The precipitate was filtered and dried in vacuum to yield a white solid (10.1 g; 91%). The white solid (2.0 g; 8.4 mmol) and NaHCO₃ (6 g; 71.4 mmol) were mixed in water (50 mL). The mixture was cooled to 0° C. Two portions of benzyl chloroformate (2×0.8 mL; 5 mmol) were added to the mixture under continuous stirring within 10 minutes of each other. The mixture was continuously stirred at room temperature for 12 h. NaHCO₃ (20 mL; 10% solution) was added to the mixture. The mixture was extracted with ether (“Et₂O”) (50 mL). Aqueous HCl (20 mL) was gradually added to the mixture and caused the formation of a bulky white precipitate. The precipitate was filtered, washed with cold water, and dried in vacuum to yield 19 as a white solid (2.483 g; 79%).

Step b. Dry CH₂Cl₂ (10 mL) and 3 (0.27 g; 0.76 mmol) were mixed. Triisopropylsilane (0.16 mL; 0.76 mmol) was added to the mixture via a syringe. Trifluoroacetic acid (0.56 mL; 7.6 mmol) was added drop wise to the mixture. The mixture was continuously stirred at room temperature for 8 h. The reaction was quenched with a saturated NaHCO₃ solution and the product was extracted with CH₂Cl₂ (2×25 mL). The organic layer was dried over anhydrous Na₂SO₄ and filtered. The solvent was removed in vacuo. The crude product was purified by flash column chromatography, eluting with a hexane/EtOAc solution (1:4), to yield the free amine as a pale yellow solid (0.15 g; 80%). The solid was dissolved in dry CH₃CN (15 mL). Anhydrous Na₂CO₃ (0.93 g; 8.81 mmol) was added to the mixture. The mixture was cooled to 0° C. Bromoacetyl bromide (0.60 mL; 6.92 mmol) in CH₃CN (5 mL) was added drop wise to the mixture. The mixture was continuously stirred at room temperature for 12 h. The mixture was diluted with EtOAc (10 mL), stirred for 1 h, and filtered. The filtrate was concentrated, redissolved in EtOAc (10 mL), and re-concentrated in vacuum to yield a pale yellow solid (2.28 g; 96%). The pale yellow solid (0.19 g; 0.59 mmol) and MeOH (5 mL) were mixed. The pale yellow solid/MeOH mixture was added drop wise to a stirred solution of ammonia in MeOH (5 mL; 0.7 M) at 0° C. The mixture was continuously stirred at room temperature for 6 h. Excess ammonia was removed in vacuum. The crude product was purified by flash column chromatography, eluting with a CH₂Cl₂/MeOH solution (4:1), to yield 20 as a pale yellow solid (0.13 mg; 81%).

Step c. Dry THF (2 mL) and 19 (31 mg; 0.082 mmol) were mixed. CDMT (33 mg; 0.18 mmol) was added to the mixture at 0° C. NMM (0.02 mL; 0.18 mmol) in THF (0.1 mL) was added drop wise to the mixture. The mixture was continuously stirred at 0° C. for 12 h. In a separate flask 20 (56 mg; 0.18 mmol) was dissolved in a THF/DMF solution (1 mL; 1:1). NMM was added to the flask (0.02 mL; 0.18 mmol). The contents of the flask was added to the first mixture under continuous stirring at 0° C. The mixture was continuously stirred for 20 h with the reaction slowly warming to room temperature. The reaction was quenched with an aqueous HCl solution (0.1 N) added drop wise under stirring and the compound was extracted with EtOAc (25 mL). The organic layer was dried over anhydrous Na₂SO₄ and filtered. The solvent was removed in vacuum. The crude product was purified by flash column chromatography, eluting with a CH₂Cl₂/MeOH solution (93:7), to yield 21 as a white solid (62 mg; 78%).

Step d. CuSO₄ (31 mg; 0.125 mmol) in a water/THF solution (1:1; 10 mL), 21 (50 mg; 0.052 mmol), 9 (175 mg; 0.229 mmol), and sodium ascorbate (50 mg, 0.25 mmol) were mixed. The mixture was continuously stirred at room temperature for 36 h. The solvent was evaporated. The crude product was purified by column chromatography, eluting with a CH₂Cl₂/MeOH solution (80:20), to yield a white solid (122 mg; 58%).

Step e. The white solid was dissolved in MeOH (20 mL). NaOMe in MeOH (0.7 M; 0.5 mL) was added drop wise to the mixture. The mixture was continuously stirred at room temperature for 12 h. The solvent was removed in vacuum. The white solid was dissolved in aqueous NaOH (3 mL; 0.05 N) and continuously stirred at room temperature for 10 h. The reaction was quenched with a careful addition of Amberlite H+ resin (pH≈6). The resin was filtered and the solvent was removed in vacuum. The crude product was purified by size exclusion chromatography using Biogel P-2 gel to yield 22 as a pure white solid (27 mg; 85%).

Step f. Biotin (0.12 g; 0.49 mmol) and CDMT (0.10 g; 0.56 mmol) is mixed in a dry THF/DMF solution (1:1; 6 mL) under argon at 0° C. NMM (0.11 mL) in THF (1.0 mL) is added drop wise to the mixture. The mixture is continuously stirred at 0° C. for 12 h. In a separate flask the free amine (48 mg; 0.027 mmol) is dissolved in a DMF/THF solution (1:1; 1 mL). NMM (0.11 mL) is added to the flask. The flask mixture is added to the activated biotin at 0° C. and the mixture is continuously stirred for 20 h with the reaction slowly warming to room temperature. The reaction is quenched with an aqueous HCl solution (0.1 N) added drop wise and the compound is extracted with EtOAc. The organic layer is dried over anhydrous Na₂SO₄ and filtered. The solvent is removed in vacuo. The crude product is purified by flash column chromatography too yield 23.

It is understood that the foregoing detailed description and Examples are merely illustrative and are not to be taken as limitations upon the scope of the invention, which is defined by the appended claims. Various changes and modifications to the disclosed embodiments will be apparent to those skilled in the art. Such changes and modifications, including without limitation those relating to syntheses, formulations, and/or methods of use of the invention, may be made without departing from the spirit and scope thereof.

All publications, patents, and patent applications cited in this specification are herein incorporated by reference as if each individual publication or patent application were specifically and individually indicated to be incorporated by reference.

We claim

1. A compound of structure

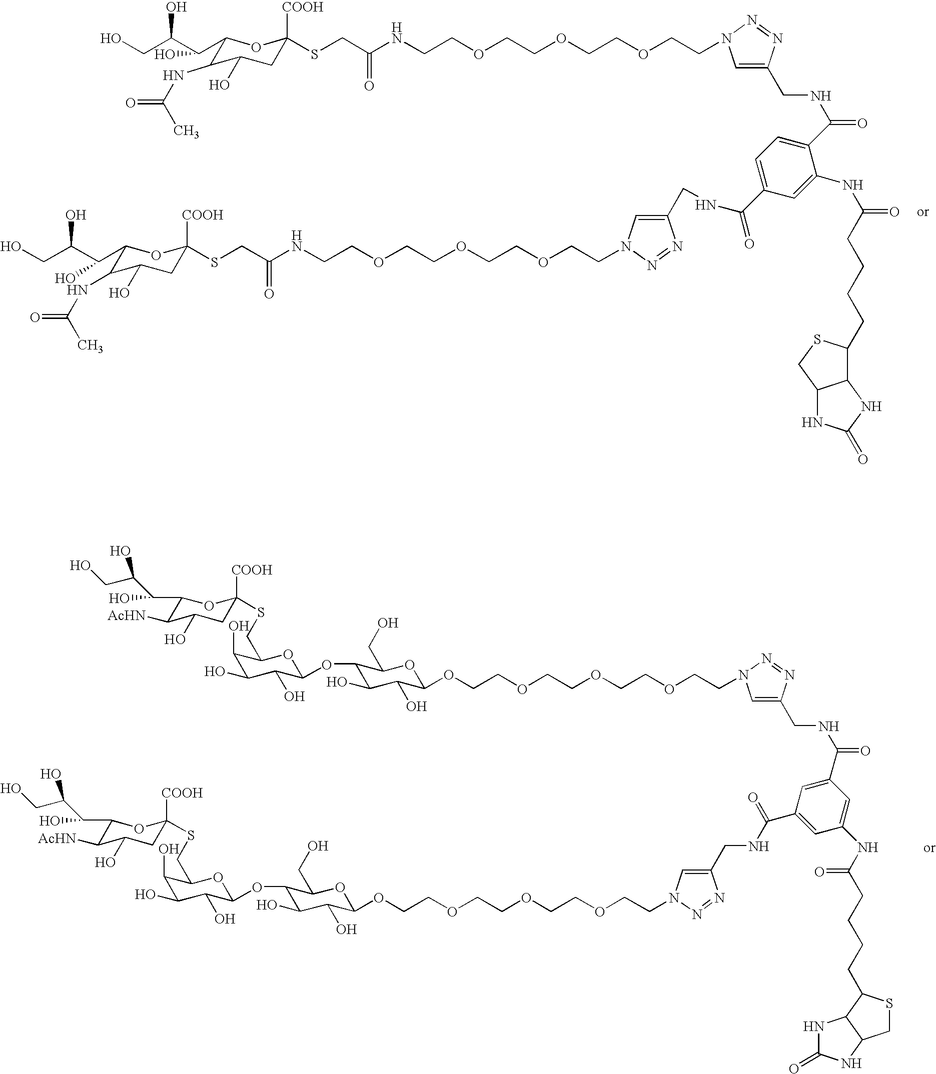

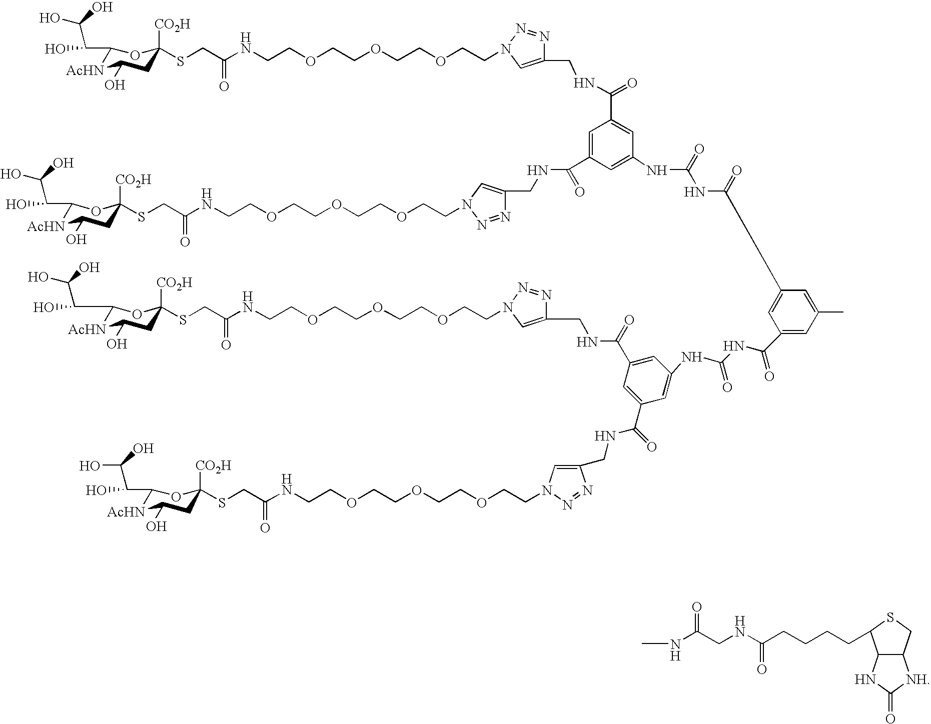

2. A compound of structure 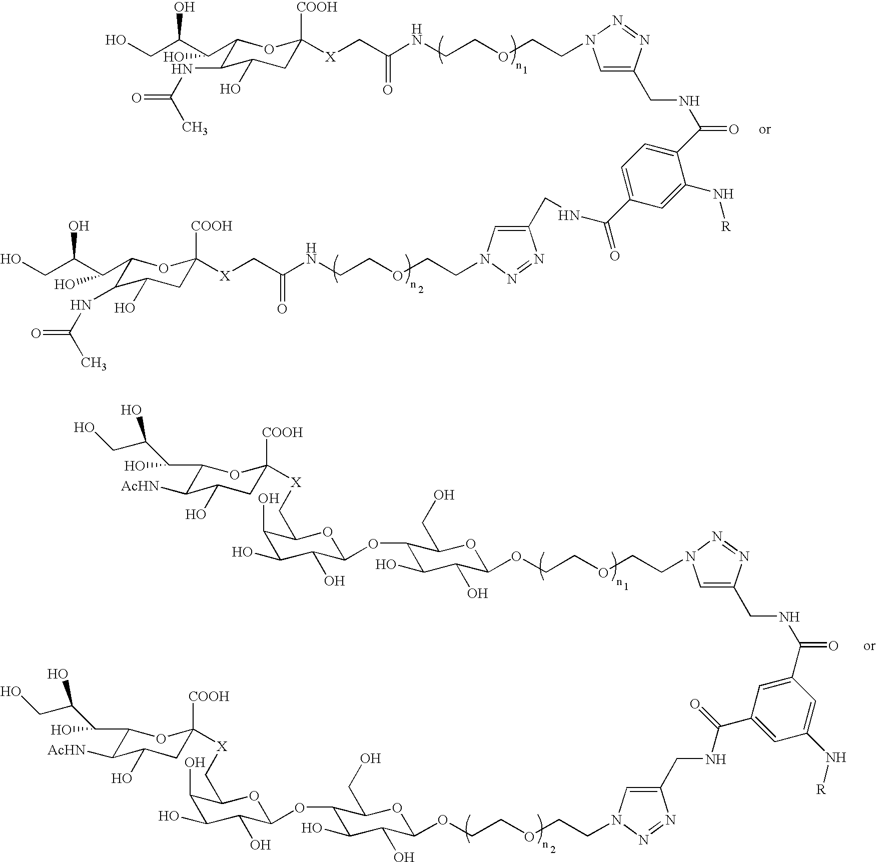 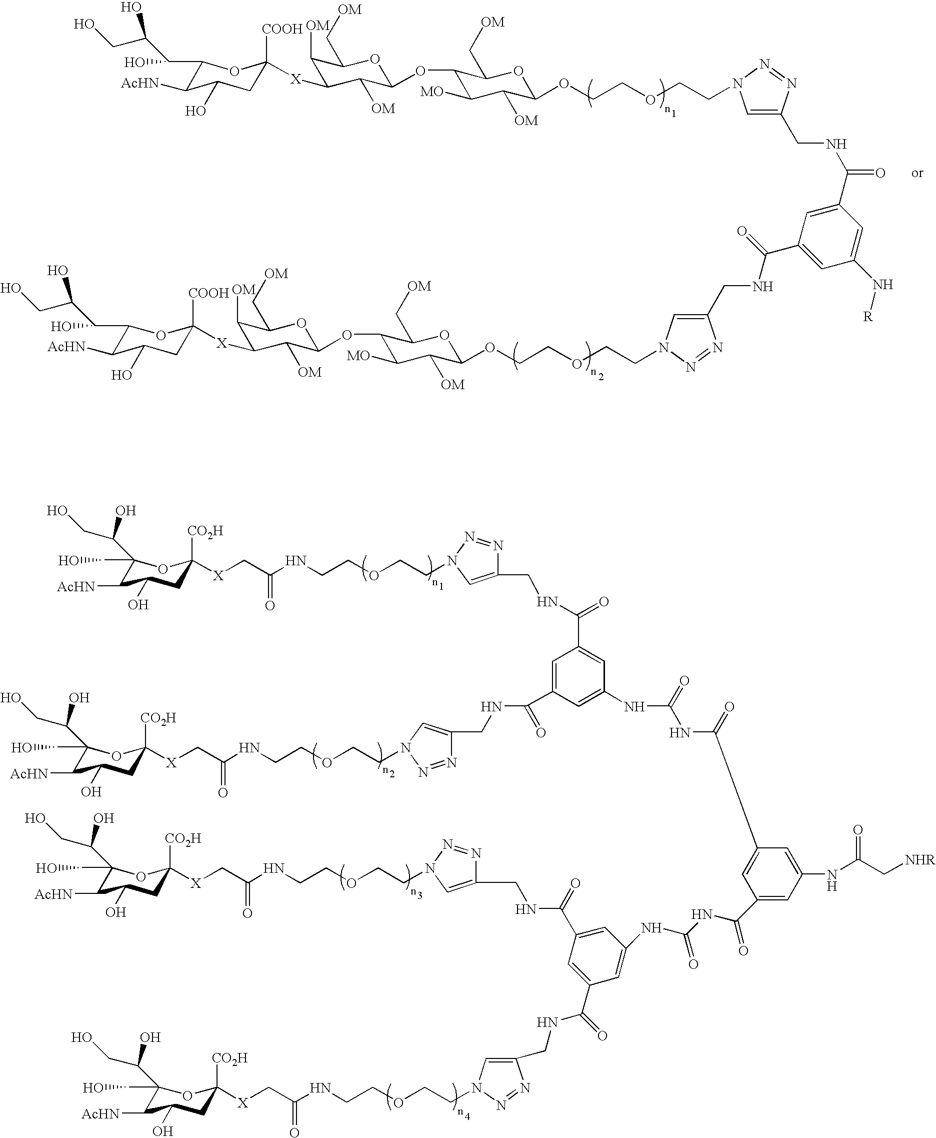
wherein
R is a functional group capable of either
(a) attaching to a substrate, membrane, or magnetic bead, or
(b) providing an output signal;
n₁, n₂, n₃, and n₄ are integers from 3 to 21;
M is a functional group independently selected from the group consisting of an amine, a guanidium group, a sulfate, a carbohydrate, and a peptide; and
X is independently selected from the group consisting of —S—, —S(═O)—, —SO₂—, —NH—, —NA- wherein A is an alkyl, —CH₂—, —CHA-, —CA₂-, and —C(═O)—.

3. The compound of claim 2 wherein R is attached to a membrane, a self-assembled monolayer, a waveguide, a magnetic bead, a protein, a solid phase, or an anchor.

4. A compound of structure 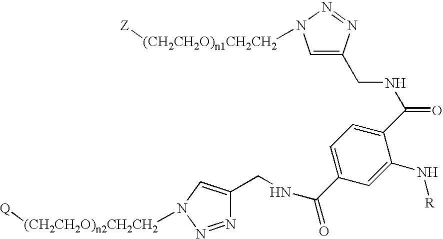
wherein
R is a functional group capable of either
(a) attaching to a substrate, membrane, or magnetic bead, or
(b) providing an output signal; and
n₁ and n₂ are integers from 3 to 21.
Z and Q are independently selected from materials capable of attaching to a hemagglutinin or a neuraminidase.

5. The compound of claim 4 wherein said hemagglutinin or said neuraminidase is attached to an intact organism.

6. The compound of claim 4 wherein Z and Q are independently selected from the group consisting of 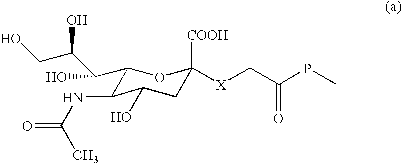
wherein
X is independently selected from the group consisting of —S—, —NH—, —CH—, —C(═O)—,
P is independently selected from the group consisting of —O— and —NH—; and

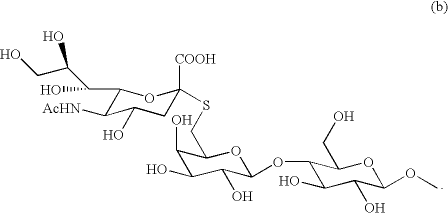

7. The compound of claim 6 wherein

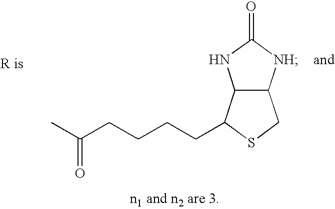

8. The compound of claim 4 wherein R is attached to a membrane, a self-assembled monolayer, a waveguide, a magnetic bead, a protein, a solid phase, or an anchor.

9. A compound comprising:
(a) a flexible spacer having a first terminus and a second terminus;
(b) an attachment element connected to said first terminus and comprising a di-, tri-, tetra, or multivalent scaffold and that is capable of either (i) providing an output signal or (ii) attaching to a substrate, membrane, or a magnetic bead; and
(c) a recognition element connected to said second terminus that is capable of attaching to (i) a hemagglutinin, (ii) a neuraminidase, or (iii) a hemagglutinin or a neuraminidase attached to an intact organism.
